(19) United States
(12) Patent Application Publication
(10) Pub. No.: US 2011/0144659 A1
(43) Pub. Date: Jun. 16, 2011

(54) N DEGREES-OF-FREEDOM (DOF) LAPAROSCOPE MANEUVERABLE SYSTEM

(75) Inventor: Mordehai Sholev, Amikam (IL)

(73) Assignee: M.S.T. MEDICAL SURGERY TECHNOLOGIES LTD., Caesarea (IL)

(21) Appl. No.: 13/058,901

(22) PCT Filed: Aug. 13, 2009
(86) PCT No.: PCT/IL09/00800

§ 371 Date: Feb. 14, 2011

Related U.S. Application Data
(60) Provisional application No. 61/088,765, filed on Aug. 14, 2008.
(60) Provisional application No. 61/171,849, filed on Apr. 23, 2009.

(51) Int. Cl. A61B 19/00

(52) U.S. Cl. 606/130

(57) ABSTRACT

A laparoscope including a cylindrical device of multiple degrees of freedom which can be inserted through a small surgical incision. This is accomplished by means of a series of coaxial members nested within the aforementioned cylinder, each can rotate independently and actuate a desired motion at the distal end. The laparoscope has multiple consecutive arm sections, each includes several coaxial input shafts adapted to be rotated around an input axis of rotation by multiple sources of torque. In addition, several constant velocity couplers connect the arm sections and are equipped with coaxial input transmission means, coaxial second transmission means and coaxial output transmission means to transfer the input torque to coaxial output shafts and facilitate the independent rotation and motion of the device distal end within the patient's body.

### FIELD OF THE INVENTION

The present invention relates to a method and apparatus for a laparoscopic surgery using a novel torque transmission member.

### BACKGROUND OF THE INVENTION

In laparoscopic surgery, the surgeon performs an inspection or operation through a small incision. This is generally accomplished using long, thin instruments adapted to be inserted through the small incision, and to be moved about in a body cavity. These instruments are generally provided with imaging means for observing the internal cavity. The laparoscope is often provided with several degrees of freedom to allow relatively complex procedures to be performed within the body. Generally these degrees of freedom are achieved by rotating and translating slender members inserted through the incision, using motoring means or the like. In the more successful examples, such members are attached to gantries that hold the part of the laparoscope outside the body (aka the proximal portion) still in one or more dimensions. It will be appreciated that providing additional degrees of freedom to such a device will generally involve increased complexity of the device itself, its holder, and the motoring means. Furthermore, with the development of ever more complex laparoscopic instruments and the desire to perform more complex surgeries laparoscopically, the provision of an ever greater number of degrees of freedom can be expected.

Hence, a system for laparoscopic surgery providing many degrees of freedom in a simplified fashion is still a long felt need.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which

FIG. 1A-D present a Universal Joint, also known as the U-joint or Cardan joint;

FIG. 2A-B present a constant-velocity or CV joint;

FIG. 3 presents a Thompson joint, this being a type of double Cardan joint;

FIG. 4a,b presents an embodiment of the variable coupling of the present invention in realistic and outline views, respectively;

FIG. 5a,b, presents an embodiment of the variable coupling of the present invention in realistic and outline views, respectively;

FIG. 6 presents an isometric view of a second embodiment of the variable coupling of the present invention;

FIG. 7 presents a different isometric view of the second embodiment of the variable coupling of the present invention;

FIG. 8 presents a series of three of the variable couplings of the present invention in series;

FIGS. 9A-9C presents prior art laparoscope positioning devices;

FIG. 10A, B presents a laparoscopic arm based on the coupling of the current invention.

FIG. 11A, B presents further views of a laparoscopic instrument based on the coupling of the current invention.

FIG. 12A, B presents further views of a laparoscopic instrument based on the coupling of the current invention.

FIG. 13A, B presents further views of a laparoscopic instrument based on the coupling of the current invention.

FIG. 14A, B presents a surgical procedure using a robotic laparoscopic arm based on the coupling of the current invention.

FIG. 15 presents another surgical procedure using a robotic laparoscopic arm based on the coupling of the current invention.

FIG. 16 presents further views of a surgical procedure using a robotic laparoscopic arm based on the coupling of the current invention.

FIG. 17A, B presents further views of a surgical procedure using a robotic laparoscopic arm based on the coupling of the current invention.

FIG. 18 presents a laparoscopic instrument based on the coupling of the current invention provided with attachment straps.

FIG. 19 presents another view of a laparoscopic instrument based on the coupling of the current invention provided with attachment straps.

FIG. 20 presents another view of a laparoscopic instrument based on the coupling of the current invention in use during surgery provided with arm attachment straps.

FIG. 21 presents a laparoscopic instrument based on the coupling of the current invention in use during surgery provided with thigh attachment straps.

FIG. 22 presents a laparoscopic instrument based on the coupling of the current invention in use during surgery provided with leg attachment straps.

FIG. 23A-23G illustrates another embodiment of the present invention in which a non motorized laparoscope/endoscopes maneuvering system is provided.

### SUMMARY OF THE INVENTION

It is an object of the invention to provide a laparoscope including a cylindrical device of multiple degrees of freedom which can be inserted through a small surgical incision. This is accomplished by means of a series of coaxial members nested within the aforementioned cylinder, each of which can rotate independently and actuate a desired motion at the distal end. It is an integral object of the invention to provide a novel joint that allows two such cylindrical devices to be mated while transmitting the rotations of the coaxial members, and allowing the two cylindrical devices to be pivoted with respect to one another.

It is one object of the present invention to provide a p degrees-of-freedom (DOF) laparoscope maneuverable system comprising:
a. k consecutive arm sections, each comprising n coaxial input shafts adapted to be rotated around an input axis of rotation by m sources of torque, where n and m and k are positive integers;
b. at least k−1 constant velocity couplers coupling each two of said k consecutive arm sections together, each of said constant velocity coupler comprising:
   i. n coaxial input transmission means, each of which is coupled to one of said n input shafts; said input transmission means defining a first plane substantially perpendicular to said input axis of rotation;
   ii. n coaxial second transmission means rotatably connected to said n input transmission means; said second transmission means rotating in a second plane, such that said second plane is substantially perpendicular to said first plane;
   iii. n coaxial output transmission means rotatably connected to said n second transmission means; said output transmission means rotating in a third plane; said third plane being substantially perpendicular to said second plane;
b. n coaxial output shafts, each of which is coupled to one of said n output transmission means, said n output shafts being adapted to rotate around an output axis of rotation; such that (i) turning a given input shaft at a constant velocity will provide a constant velocity at the corresponding output shaft; and, (ii) the angle between said input axis of rotation and said output axis of rotation varies in said second plane in an angular range of about 0 to about 360 degrees;
c. at least one laparoscope coupled to at least one of said k consecutive arm sections;
   wherein said p DOF are at least 7 DOF provided to said k consecutive arm sections such that said laparoscope is maneuvered.

It is another object of the present invention to provide the laparoscope maneuverable system as defined above, wherein said 7 DOF are selected from a group consisting of at least 6 rotation movements (1007, 1009, 1010, 1011, 1012, 1013, 1601, 1602), at least 1 translation movements (1008) or any combination thereof.

It is another object of the present invention to provide the laparoscope maneuverable system as defined above, wherein said input transmission means, second transmission means, and said output transmission means are selected from a group consisting of gearwheels, wheels, crown gears, bevel gears, spur gears, belts, and combinations thereof.

It is another object of the present invention to provide the laparoscope maneuverable system as defined above, additionally comprising:
a. an axial support member (601) adapted to provide axial support to said n output shafts in said third plane; and,
b. a circular track (618) centered on the axis of rotation of said second transmission means, said axial support member being adapted to fit into said track and slide within it.

It is another object of the present invention to provide the laparoscope maneuverable system as defined above, additionally comprising a radial support member (604) adapted to provide radial support to said n output shafts, said radial support member being adapted to rotate in said second plane.

It is another object of the present invention to provide the laparoscope maneuverable system as defined above, wherein the gear ratio between said input and output shafts is between about 10 and about 0.1.

It is another object of the present invention to provide the laparoscope maneuverable system as defined above, additionally comprising n coaxial auxiliary shafts in rotating communication with said n second transmission means, said n coaxial auxiliary shafts rotating in said second plane, and said n coaxial auxiliary shafts capable of either being driven by said input shafts or driving said input shafts.

It is another object of the present invention to provide the laparoscope maneuverable system as defined above, additionally comprising locking means adapted for preventing relative movement between one or more of said input axis shafts and said constant velocity joint, wherein said constant velocity joint is caused to rotate as a body with said locked input axis shafts.

It is another object of the present invention to provide the laparoscope maneuverable system as defined above, comprising locking means for preventing relative movement between one or more of said output axis shafts and said constant velocity joint, wherein said constant velocity joint is caused to rotate as a body with said locked output axis shafts.

It is another object of the present invention to provide a method of maneuvering surgical instruments during laparoscopic surgery whilst providing p Degrees of Freedom (DOF). The method comprising steps selected inter alia from:
a. providing k consecutive arm sections, each comprising n coaxial input shafts adapted to be rotated around an input axis of rotation by m sources of torque, where n and m are positive integers;
b. providing at least k−1 constant velocity couplers coupling said consecutive arm sections, each said constant velocity coupler adapted to be rotated around an input axis of rotation by m sources of torque, where n and m are positive integers and each said constant velocity coupler comprising:
   i. n coaxial input transmission means, said input transmission means defining a first plane substantially perpendicular to said coaxial axis;
   ii. n coaxial second transmission means rotatably connected to said n input transmission means; said second transmission rotating in a second plane, such that said second plane is substantially perpendicular to said first plane;
   iii. n coaxial output transmission means rotatably connected to said n second transmission means; said output transmission means rotating in a third plane; said third plane being substantially perpendicular to said second plane;
c. providing n coaxial output shafts, said n output shafts being adapted to rotate around an output axis of rotation, and said n output shafts being coupled to said n coaxial output transmission means, whereby turning a given input shaft at a constant velocity will provide a constant velocity at the corresponding output shaft, and furthermore wherein the angle between said input axis of rotation and said output axis of rotation varies in said second plane in an angular range of about 0 to about 360 degrees;
d. coupling said k consecutive arm sections together with said k−1 couplers;
e. providing at least one surgical instrument;
f. coupling said surgical instrument to said output shafts; and,
g. maneuvering said surgical instrument by means of rotating one or more of said coaxial input shafts thereby providing said p DOF to said k consecutive arms and said surgical instrument,
   wherein said step of proving said p DOF comprises at least 7 DOF provided to said k consecutive arm sections.

It is another object of the present invention to provide a method for transmitting torque to surgical instruments during laparoscopic surgery. The method comprising steps selected inter alia from:
a. providing k consecutive arm sections, each comprising n coaxial input shafts adapted to be rotated around an input axis of rotation by m sources of torque, where n and m are positive integers;
b. providing at least k−1 constant velocity couplers coupling said consecutive arm sections together, said constant velocity couplers comprising:
   i. n coaxial input transmission means, each of which is coupled to one of said n input shafts; said input transmission means rotating in a first plane substantially perpendicular to said coaxial axis;
   ii. n coaxial second transmission means rotatably connected to said n input transmission means; said second transmission means rotating in a second plane, such that said second plane is substantially perpendicular to said first plane;
   iii. n coaxial output transmission means rotatably connected to said n second transmission means; said output transmission means rotating in a third plane; said third plane being substantially perpendicular to said second plane;
c. n coaxial output shafts, each of which is coupled to one of said n output transmission means, said n output shafts being adapted to rotate around an output axis of rotation, whereby turning a given input shaft at a constant velocity will provide a constant velocity at the corresponding output shaft, and furthermore wherein the angle between said input axis of rotation and said output axis of rotation varies in said second plane in an angular range of about 0 to about 360 degrees;
d. providing at least one surgical instrument;
e. coupling said surgical instrument to said output shafts; and,
f. performing surgical functions by means of rotating one or more of said coaxial input shafts thereby providing said p DOF to said k consecutive arms and said surgical instrument;
   wherein said step of proving said p DOF comprises at least 7 DOF provided to said k consecutive arm sections.

It is another object of the present invention to provide the methods as defined above, additionally comprising step of selecting said 7 DOF from a group consisting of at least 6 rotation movements (1007, 1009, 1010, 1011, 1012, 1013, 1601, 1602), at least 1 translation movements (1008) or any combination thereof.

It is another object of the present invention to provide the methods as defined above, wherein said input transmission means, second transmission means, said output transmission means are selected from a group consisting of gearwheels, wheels, crown gears, bevel gears, spur gears, belts, or any combination thereof.

It is another object of the present invention to provide the methods as defined above, additionally comprising
a. an axial support member (601) adapted to provide axial support to said n output shafts in said third plane; and,
b. a circular track (618) centered on the axis of rotation of said second transmission means, said axial support member being adapted to fit into said track and slide within it.

It is another object of the present invention to provide the methods as defined above, additionally comprising a radial support member (604) adapted to provide radial support to said n output shafts, said radial support member being adapted to rotate in said second plane. It is another object of the present invention to provide the methods as defined above, providing a gear ratio between said input and output shafts is between about 10 and about 0.1.

It is another object of the present invention to provide the methods as defined above, additionally comprising the step of providing n coaxial auxiliary shafts in rotating communication with said n second transmission means, said n coaxial auxiliary shafts rotating in said second plane, and said n coaxial auxiliary shafts either being driven by said input shafts or driving said input shafts.

It is another object of the present invention to provide the methods as defined above, comprising locking means adapted for preventing relative movement between one or more of said input axis shafts and said constant velocity joint, wherein said constant velocity joint is caused to rotate as a body with said locked input axis shafts.

It is another object of the present invention to provide the methods as defined above, additionally comprising locking means adapted for preventing relative movement between one or more of said output axis shafts and said constant velocity joint, wherein said constant velocity joint is caused to rotate as a body with said locked output axis shafts.

It is another object of the present invention to provide a laparoscopic instrument characterized by p degree-of-freedom (DOE) comprising:
a. k consecutive arm sections, each comprising n coaxial input shafts adapted to be rotated around an input axis of rotation by m sources of torque, where n and m are positive integers;
b. at least k−1 constant velocity couplers coupling each two of said consecutive arm sections together, each of said constant velocity couplers comprising:
   i. n coaxial input transmission means, each of which is coupled to one of said n input shafts; said input transmission means defining a first plane substantially perpendicular to said input axis of rotation;
   ii. n coaxial second transmission means rotatably connected to said n input transmission means; said second transmission means rotating in a second plane, such that said second plane is substantially perpendicular to said first plane;
   iii. n coaxial output transmission means rotatably connected to said n second transmission means; said output transmission means rotating in a third plane; said third plane being substantially perpendicular to said second plane;
c. n coaxial output shafts, each of which is coupled to one of said n output transmission means, said n output shafts being adapted to rotate around an output axis of rotation, whereby turning a given input shaft at a constant velocity will provide a constant velocity at the corresponding output shaft, and furthermore wherein the angle between said input axis of rotation and said output axis of rotation varies in said second plane in an angular range of about 0 to about 360 degrees;
d. at least one laparoscopic instrument coupled to at least one of said k consecutive arm sections, adapted for performing surgical functions;
wherein said p DOF comprise at least 7 DOF provided to said surgical tools

It is another object of the present invention to provide a non motorized laparoscopic/endoscopic maneuvering device comprising:
a. at least two consecutive arm section;
b. at least one gimbal at least coupling each two of said consecutive arm sections together; and,
c. at least one laparoscope coupled to said consecutive arm;
wherein said laparoscopic/endoscopic maneuvering device maneuvers said laparoscopic/endoscopic in a non motorized manner.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide means for controlling the spatial position of endoscope tube or laparoscope in laparoscopic surgery. The present device is cheap, easily install and disassemble, comfortable to use, not limiting the dexterity of the surgeon and having small physical dimension. Most importantly the present device provides at least 7 degrees of freedom (DOF). The multiple DOF is mainly achieved due to the coupling of the system with variable axial direction of the output with respect to the input. The small size of present invention is achieved by:
1. the shape of the system;
2. the coupling of a dedicated constant velocity couplers within the system; the constant velocity couplers enables both linear zoom mechanism and a rotational mechanism that rotates the endoscope and/or the camera about ifs long axis, independently of other moving parts of the mechanism;

The inexpensive price of the present invention is achieved by:
1. the small physical dimension of the system according to present invention;
2. the simplicity of the mechanisms of the present invention;

The easy installation and disassemble processes is achieved by
1. the small physical dimension of the present invention;
2. the safety mechanisms of the present invention;
3. the movement compensation mechanisms of the present invention;

In the following detailed description, numerous specific details are set forth in order to provide a thorough understanding of embodiments of the present invention. However, those skilled in the art will understand that such embodiments may be practiced without these specific details. Reference throughout this specification to “one embodiment” or “an embodiment” means that a particular feature, structure, or characteristic described in connection with the embodiment is included in at least one embodiment of the invention.

The term ‘gear ratio’ in a transmission with an input shaft and an output shaft refers to the ratio of angular velocity of the output shaft to that of the input shaft.

The term ‘transmission means’ here refers to means for transferring torque from one rotating element to another, such as gearwheels, wheels, crown gears, and the like.

The term ‘plurality’ refers hereinafter to any integer number equal or higher 1, e.g., 2-10, especially 2-4.

The terms “endoscope” and “laparoscope” refer interchangeably hereinafter to a fiber optical device that consists of a flexible tube. Glass or plastic filaments allow the internal refraction of light for viewing. This medical device is used in laparoscope, endoscope, laparoscopic and endoscopic surgeries. It is also in the scope of the invention wherein the term refers also to any means for looking within body cavities, especially inside the human body and mammalian body for medical reasons using an instrument; and especially to means for minimally invasive diagnostic medical procedure, such as rigid or flexible endoscopes, fiberscopes, means for robotic surgery, trocars, surgical working tools and diagnosing means etc.

The terms “endoscopic surgery” and “laparoscopic surgery” interchangeably refer hereinafter to a modern surgical technique in which operations upon the body of a patient, e.g., within the abdomen, are performed through small incisions (usually 0.5 to 1.5 cm) as compared to larger incisions needed in traditional surgical procedures. Laparoscopic surgery includes e.g., operations within the abdominal, pelvic or joint cavities. Endoscopic surgery involves, inter alia, operations in the gastrointestinal tract, e.g., in the esophagus, stomach and duodenum (esophagogastroduodenoscopy), small intestine, colon (colonoscopy, proctosigmoidoscopy), bile duct, endoscopic retrograde cholangiopancreatography (ERCP), duodenoscope-assisted cholangiopancreatoscopy, intraoperative cholangioscopy, the respiratory tract, the nose (rhinoscopy), the lower respiratory tract (bronchoscopy), the urinary tract (cystoscopy), the female reproductive system, the cervix (colposcopy), the uterus (hysteroscopy), the Fallopian tubes (falloscopy), normally closed body cavities (through a small incision), the abdominal or pelvic cavity (laparoscopy), the interior of a joint (arthroscopy) organs of the chest (thoracoscopy and mediastinoscopy), the amnion during pregnancy (amnioscopy), the fetus (fetoscopy), plastic surgery, panendoscopy, laryngoscopy, esophagoscopy; and various non-medical uses for endoscopy. The term also refers to any manipulation of laparoscopes and endoscopes as defined above into the body of a patient.

The term “Degrees of freedom” (DOF) refers hereinafter to a set of independent displacements that specify completely the displaced position of the endoscope or laparoscope as defined above. In three dimension space, there are six DOF, three DOF of linear displacement and three rotational DOFs, namely, moving up and down, moving left and right, moving forward and backward, tilting up and down, turning left and right, tilting side to side. The present invention refers to a system essentially comprising means for at least seven DOF selected from any of those that will be described hereinafter.

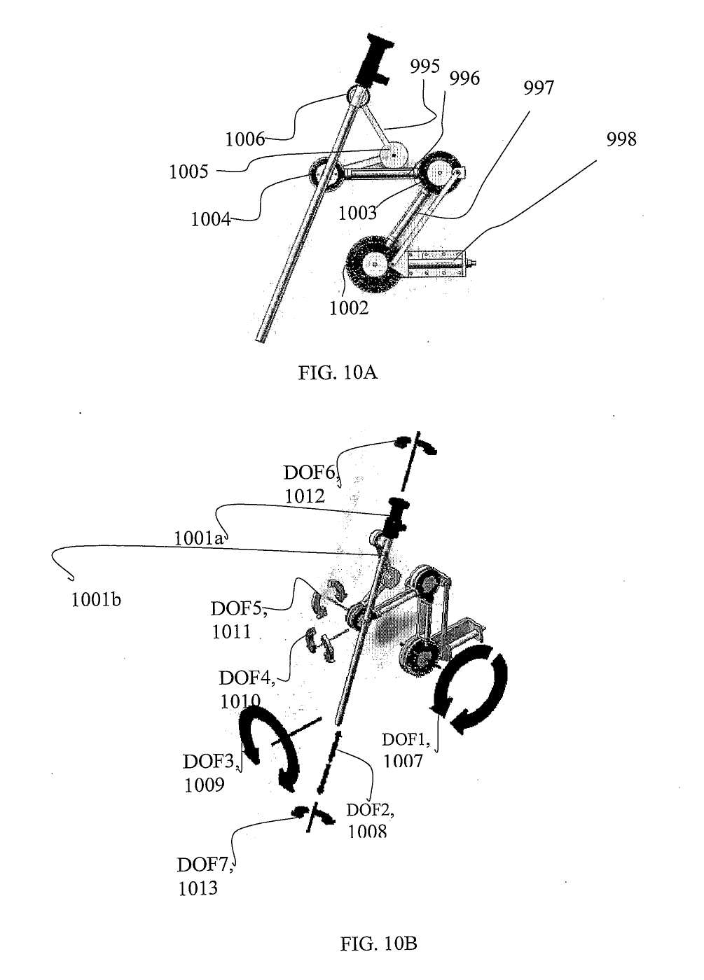

The term “seven degrees of freedom” (7DOF) refers hereinafter to a system with seven degrees of freedom, where distinct coarse and fine movements in a given direction are considered distinct degrees of freedom (the 7DOF are represented in FIG. 10). Said 7DOF are the following:

DOF1 presents the ability of the system to move the endoscope or laparoscope forward and backwards in direction represented by numerical reference 1007.

DOF2 presents the ability of the system to move the endoscope or laparoscope in a zoom movement i.e. in and out of the patient body through the penetration point (represented by numerical reference 1008).

DOF3 presents the ability of the system to move the endoscope or laparoscope to the right and left in direction represented by numerical reference 1009.

DOF4 presents the ability of the system to fine tune the endoscope or laparoscope movements to the right and to the left in direction represented by numerical reference 1010.

DOF5 presents the ability of the system to fine tune the endoscope or laparoscope movements forward and backwards in direction represented by numerical reference 1011.

DOF6 presents the ability of the system to rotate the camera 1001b with respect to the endoscope's 1001a long axis. This degree of freedom is necessary to keep the horizon of the image when using endoscope with “angled edge”.

DOF7 presents the ability of the robot to rotate the endoscope 1001b about its long axis. The terms “distal portion” and “proximal portion” refer hereinafter to the side of the endoscope within the body of the patient, and outside the body of the patient, respectively. Laparoscopic surgery, also called minimally invasive surgery (MIS), bandaid surgery, keyhole surgery, or pinhole surgery is a modern surgical technique in which operations in the abdomen are performed through small incisions (usually 0.5-1.5 cm) as compared to larger incisions needed in traditional surgical procedures. The key element in laparoscopic surgery is the use of a laparoscope, which is a device adapted for viewing the scene within the body, at the distal end of the laparoscope. Either an imaging device is placed at the end of the laparoscope, or a rod lens system or fiber optic bundle is used to direct this image to the proximal end of the laparoscope. Also attached is a light source to illuminate the operative field, inserted through a 5 mm or 10 mm cannula or trocar to view the operative field. The abdomen is usually injected with carbon dioxide gas to create a working and viewing space. The abdomen is essentially blown up like a balloon (insufflated), elevating the abdominal wall above the internal organs like a dome. Within this space, various medical procedures can be carried out.

It will be appreciated that more complex medical procedures can be carried out with a system that has a greater number of degrees of freedom. To take an extreme example, if one were to build a robotic hand with the same large number of degrees of freedom as an actual human hand, this robotic hand might in principle be capable of carrying out the same operations as an actual human hand. Thus for the performance of increasingly complex medical procedures, a system for transmitting a large number of mechanical degrees of freedom to the proximal end of a laparoscope is desirable.

The present invention solves this problem within the constraints dictated by the nature of laparoscopic surgery, namely a small incision diameter, a large distance between actuators (outside the body) and actuated elements (within the body), and the desire to provide the laparoscope with as many multiple independent degrees of freedom as possible.

The present invention solves this problem using a cylindrical device with multiple coaxial cylinders, each of which can rotate independently to actuate a desired motion at the distal end. A novel joint allows two such cylindrical devices to be mated while transmitting the rotations of the coaxial members, allowing the two cylindrical devices to be pivoted with respect to one another. The notion of concentric cylindrical members is simple enough to forego detailed discussion, and thus in the following we concentrate on the design of the joint joining two such cylindrical members.

In many mechanical systems there arises the need to transfer torque from an input shaft to an output shaft. A wide variety of gear systems have been devised for this purpose. In a number of important cases the output shaft must vary the direction of its axis with respect to the input shaft. This is the case for example in a front-wheel-drive car. The engine must provide torque to the wheels, to move the car forward. However the front wheels must also be allowed to change their axis of rotation, to allow steering of the car.

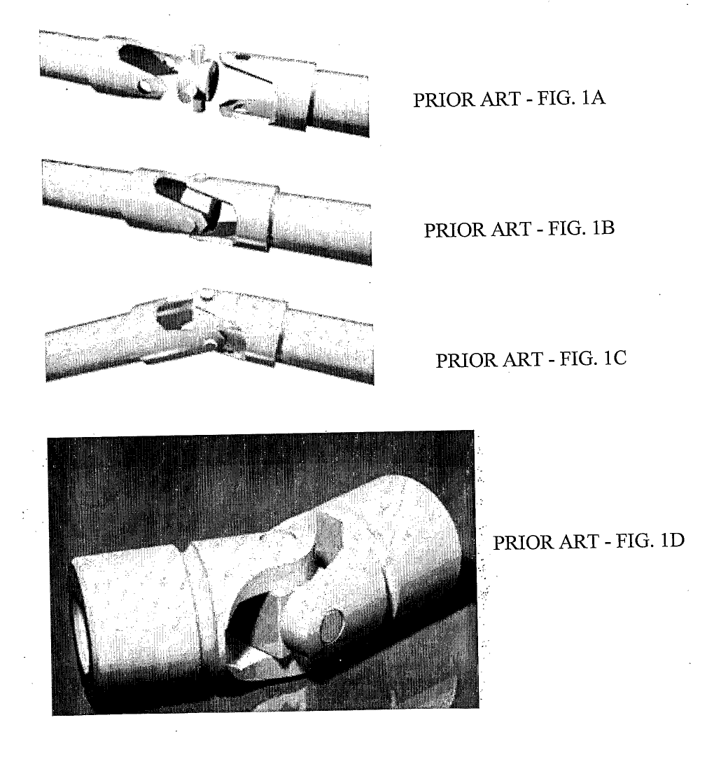

The so-called universal joint, aka U-joint, Cardan joint, Hardy-Spicer joint, or Hooke's joint is often employed for purposes of allowing variation of the output axis direction. This is a joint in a rigid rod that allows the rod to ‘bend’, and is commonly used in shafts that transmit rotary motion. It consists of a pair of ordinary hinges located close together, but oriented at 90° relative to each other. See FIG. 1a-1d for illustrations of this common joint. The concept of the universal joint is based on the design of gimbals, which have been in use since antiquity.

There are several known drawbacks to the simple U-joint. When the two shafts are at an angle other than 180° (straight), the driven shaft does not rotate with constant angular speed in relation to the drive shaft; as the angle approaches 90° the output rotation gets jerkier (and furthermore, when the shafts reach the 90° perpendicular situation, they lock and will not operate at all). We note that our measurement of angle between output and input shaft is consonant with standard mathematical practice. Namely, when the input and output shaft are parallel in the ‘unbent’ configuration, the angle between them is 180°. As the output shaft is bent, this angle decreases until reaching 90° when the shafts are perpendicular, and 0° when the output shaft is bent back upon the input shaft.

Joints have been developed utilizing a floating intermediate shaft and centering elements to maintain equal angles between the driven and driving shafts, and the intermediate shaft. This overcomes the problem of differential angles between the input and output shafts.

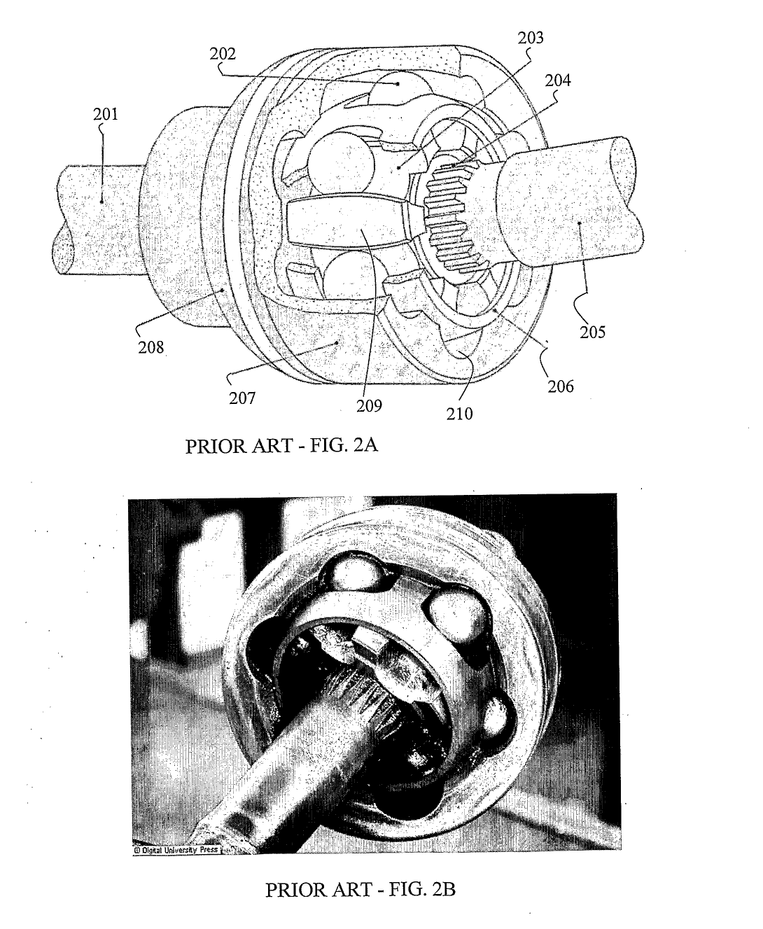

The CV joint or constant velocity joint finds actual use in automotive applications. As shown in FIG. 2 this is a joint connecting the input axle 201 to the output axle 205. The splines 204 spin the spokes 209 which in turn spin the plurality of ball bearings 202 on the inner ball race 203. These balls are confined between the ball cage 206 and the outer socket 207, which has depressions 210 into which the balls fit. Since the balls are confined by both axles, they transfer the torque from the input axle 201 to the output axle 205. An isometric view is given in FIG. 2b. The two main failures are wear and partial seizure. Furthermore it will be appreciated that extreme angles between input and output shafts of around 90 or less will not be capable of transferring torque at all, and in practice a continuous angle of about 100° degrees is the highest deviation from the straight 100° configuration obtainable with a CV joint.

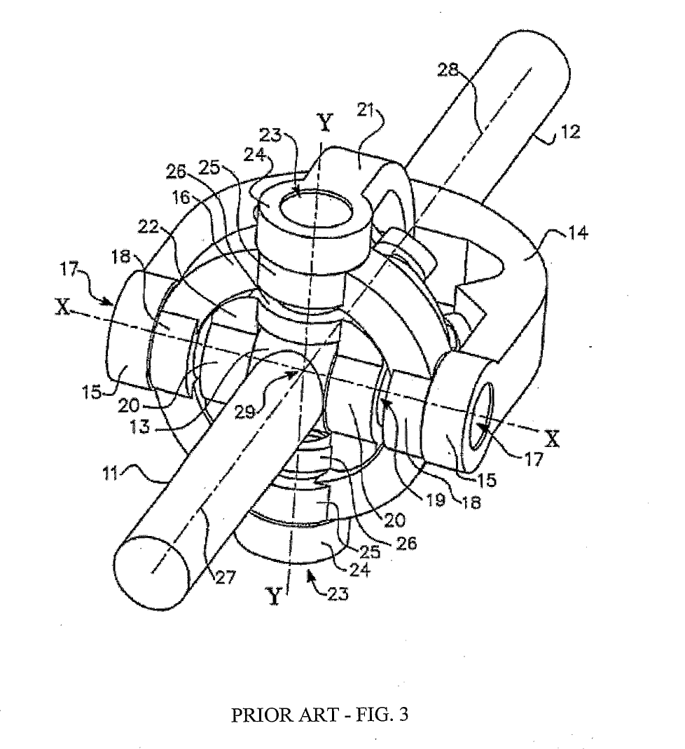

The double Cardan or double U-joint allows for a constant velocity to be attained at the output shaft, unlike the single U-joint. An improvement on this is two Cardan joints assembled coaxially where the cruciform-equivalent members of each are connected to one another by trunnions and bearings which are constrained to continuously lie on the homokinetic plane of the joint. This is the basis of US patent application 20060217206. Therein is disclosed a constant velocity coupling and control system therefore, the so-called ‘Thompson coupling’, as shown in FIG. 3. A recent innovation, the Thompson coupling is a further development of the double Cardan-joint, which doesn't rely on friction or sliding elements (as the CV joint does) to maintain a strict geometric relationship within the joint, and which is capable of transmitting torque under axial and radial loads with low frictional losses. This coupling has all loads carried by roller bearings, with no sliding or skidding surfaces whatsoever. It can tolerate axial and radial loads without degradation, with no wearing components except replaceable bearings and trunnions, and is less bulky than a double Cardan joint. However as will be appreciated from FIG. 3, this is a rather complex affair. Furthermore the maximum allowable angles are still restricted to a small range around 180°, e.g. to an instantaneous minimum allowable angle of 155° and minimum continuous angle of 168°.

According to a preferred embodiment of the present invention, a method is provided that allows the transfer of torque from an input shaft to an output shaft, whose axis of rotation may be varied continuously from nearly 0 degrees to nearly 360 degrees with respect to the axis of rotation of the input shaft.

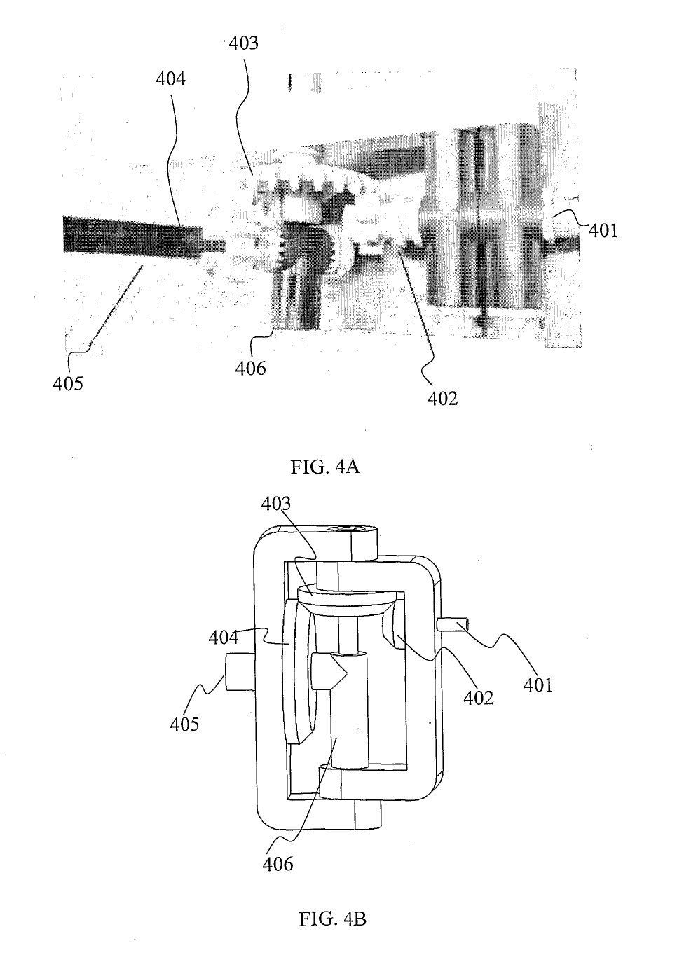

With reference to FIGS. 4a and 4b, a representative embodiment of the invention is detailed. The input shaft 401 is rotated due to torque from some external source. This torque is transmitted to spur gear 402. Spur gear 402 engages crown gear 403, which therefore rotates and transmits torque to spur gear 404. It will be appreciated by one skilled in the art that the spur and crown gears could be replaced with bevel gears. This simple arrangement is well known in the form of the bevel gear reversing mechanism. The key inventive step of the present invention is to allow the output shaft 405 to rotate not only about its own longitudinal axis but also about the axis 406. This is accomplished in the embodiment shown by coupling the output shaft 405 to axis 406 with a coupling that allows relative rotation of the output shaft 405 around axis 406. It will be appreciated that with this device, the output shaft 405 can be rotated in nearly a full circle around the axis 406 with no variation in the torque provided.

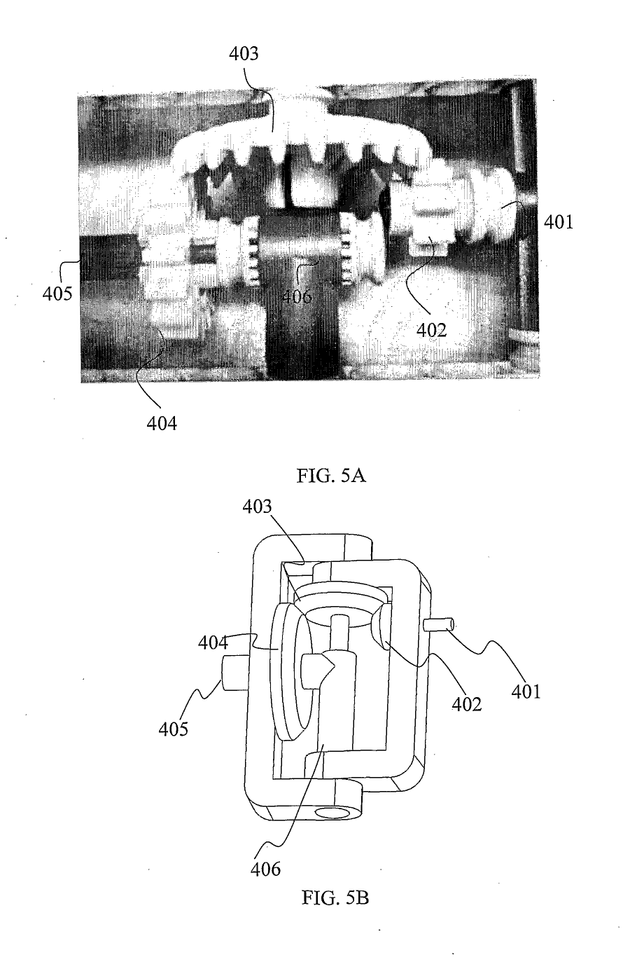

In FIGS. 5a and 5b the same embodiment is shown in plan view. Torque is transmitted from an external source to input shaft 401 and from there to gearwheel 402. Gearwheel 402 engages crown gear 403, which therefore rotates and applies torque to gearwheel 404. The output shaft 405 is thus caused to rotate. The crux of the invention lies in the extra degree of freedom allowed to the output shaft 405, namely that it may also rotate about the axis of the crown gear 403, this being the key provision of the invention. Axis 406 is preferentially but not necessarily largely collinear with the rotational axis of the planetary gear 403. Since the sizes of the gearwheels 402, 404 may be varied, the coupling as a whole can be made to provide a gear reduction or enlargement, with correspondingly greater or smaller output torque, and correspondingly smaller or greater rate of angular rotation.

It should be noted that due to the symmetry of the device, torque can also be transmitted in the opposite direction, from what we have called the output shaft to what we have called the input shaft. The terms ‘output’ and ‘input’ are therefore somewhat misleading since either can be used for output or input. Furthermore it will be appreciated that the change of the axis of rotation of output with respect to input is a relative one, and that therefore the input axis of rotation can be moved instead of the output axis of rotation, or both may be allowed to rotate with respect to a stationary coordinate system. This is more than simply a matter of nomenclature; the effect can be used for instance to transmit feedback. For example, an actuator can be used to move a certain object, and a sensor can be attached to this object such that the degree of movement achieved is transmitted back to the operator of the device. A felicitous coaxial arrangement for such an implementation requiring several simultaneous degrees of freedom is described in the following.

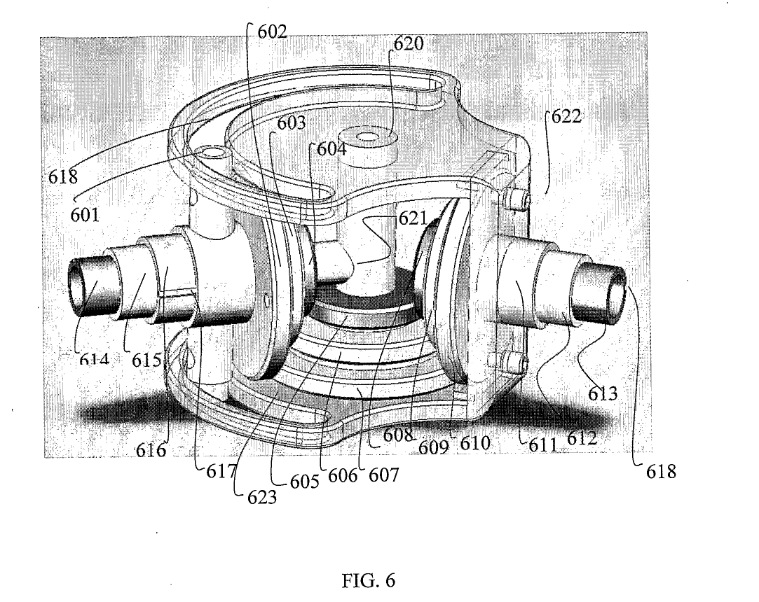

It is within the scope of the invention to allow for multiple coaxial input and output shafts to be employed simultaneously. With reference to FIGS. 6,7 an example of such an embodiment is given in isometric view. The input shafts 611,612,613 are all collinear. They may be independent or dependent, as will be determined by the configuration of keyways and shafts such as 617,618 that can couple two input shafts or two output shafts such that they rotate together. The output shafts 614,615,616 are rigidly coupled to output couplings 604,603,602 respectively and therefore rotate with them. These output couplings are caused to rotate by means of crown couplings 605,606,607 respectively. The crown couplings are caused to rotate by means of input couplings 608,609,610 respectively. These input couplings are rigidly attached to input shafts 611,612,613 and therefore rotate with them. The key provision of the invention lies in the ‘extra’ degree of freedom available to the output shafts 614,615,616 which can rotate along with output couplings 604,603,602 around the axis 620. The axial support pin 601 fits into track 618 and travels with the output shafts, supporting them against axial loading. The radial support pin 621 supports the output shafts against radial loading.

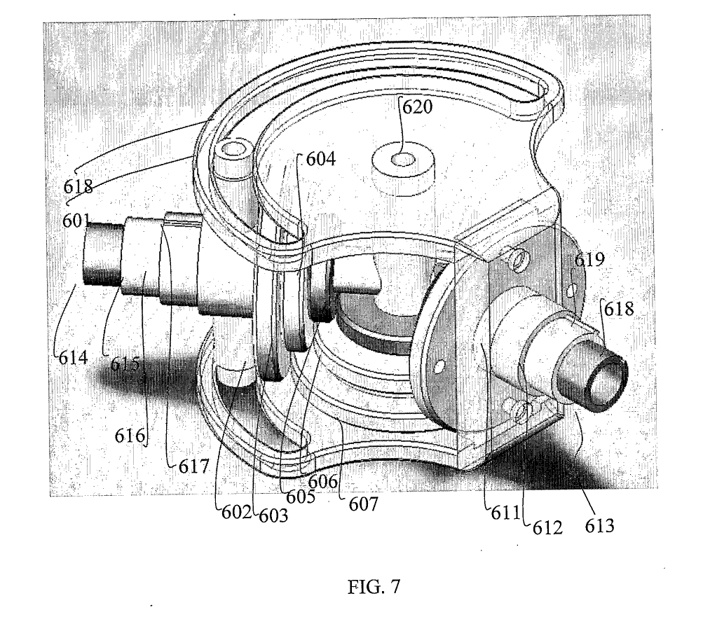

With reference to FIG. 7 the same example is shown from a slightly different angle. In this figure one can more easily see the output shafts 614,615,616 which are rigidly coupled to output couplings 604,603,602 respectively. Also more visible are the contact between these output couplings and the crown couplings 605,606,607. Also more visible here are shaft and keyway 618, 619 which couple several of the input shafts together.

A further provision of the invention is for locking of individual axes. Going back to FIG. 6 one sees that bolts 622 have been introduced which lock the outermost input shaft to the body of the coupling. Therefore any attempt to rotate this input shaft will result in a rotation of the entire coupling. In FIG. 7 it will be observed that these bolts have been removed, allowing the input shaft to move freely. Similar bolts can be added to the output shafts as well, allowing the coupling to be rotated around the axis of the output shaft. Finally, the crown couplings 605,606,607 can also be locked to the base 623 of the device. By so doing, the direction of the output shafts can be changed, as can the disposition of the entire joint itself.

It is within provision of the invention that the aforementioned bolts be replaced with coupling elements such as linear actuators, electromagnets, and the like. It will be obvious to one skilled in the art that such coupling elements can be so constructed that they couple or decouple electronically, allowing a further level of control over the device.

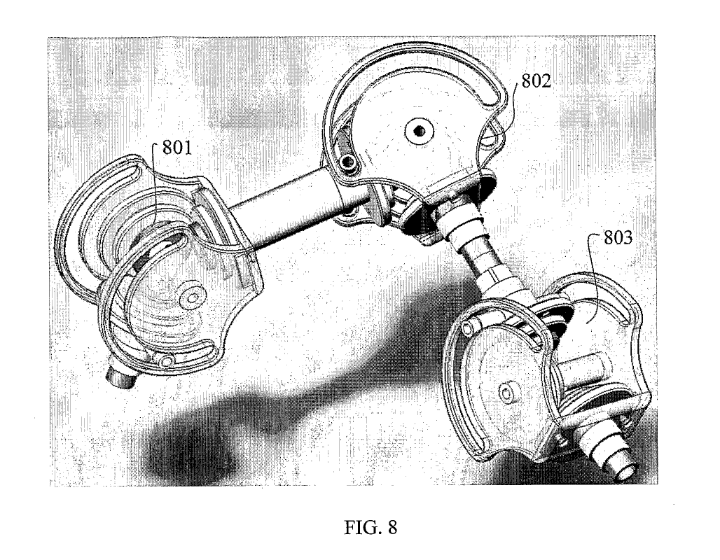

It will be noted by the astute observer that the output axis of rotation of the instant invention can rotate in a single plane only if one does not use the aforementioned provision of bolts to allow for rotation of the coupling mechanism itself. However as will be clear to one versed in the art, this restriction can be removed by the simple expedient of providing one or more further identical joints of the instant invention in series with the first, as shown in FIG. 8, where three joints 801,802,803 have been coupled in series. An embodiment with two or more joints in series provides a nearly full range of motion of the output shaft, in all directions relative to the input shaft. The only restriction on the angles is that the various shafts cannot physically overlap any other shaft, thus eliminating certain configurations from the realm of possibility. It will be appreciated however that the disallowed positions form a small proportion of the total universe of possibilities. This is especially relevant when considering that the possible input-output angles of e.g. single or double Cardan joints are restricted to small angles of around 168 degrees or less.

It will be appreciated that the gear ratio between input and output shafts can be varied by variation of the size of the wheels or gearwheels of the couplings. In particular, if the input and output gearwheels have radii r₁, r₃ then the total gear ratio will be r₁/r₃.

The constant velocity joint of the instant invention comprises:
i. An input shaft adapted to be rotated around an input axis of rotation (the longitudinal axis of the shaft) by a sources of torque.
ii. An input transmission means, coupled to one of said input shaft, said input transmission means defining a first plane substantially perpendicular to said input axis of rotation. The input transmission means may for instance be a spur gear.
iii. A second transmission means rotatably connected to said input transmission means; said second transmission means defining a second plane, such that said second plane is substantially perpendicular to said first plane. The second transmission means may comprise for instance a crown gear meshing with the first spur gear.
iv. An output transmission means rotatably connected to said second transmission means; said output transmission means defining a third plane; said third plane being substantially perpendicular to said second plane. The output transmission means may comprise for instance a spur gear meshing with the second transmission crown gear.
v. An output shaft, coupled to said output transmission means, adapted to rotate around an output axis of rotation, said axis of rotation being free itself to rotate.

It will be noted that the angle between said first input axis of rotation and said final output axis of rotation may vary in an angular range of about 0 to about 360 degrees.

The transmission means may be selected from a group consisting of gearwheels, wheels, crown gears, bevel gears, or other means for transmitting rotational motion, or combinations thereof.

In one embodiment of the invention an axial support member (601) is provided, to provide axial support to the output shafts. Also a circular track (618) centered on the axis of rotation of said second transmission means is provided, said axial support member being adapted to fit into said track and slide within it.

In one embodiment of the invention a radial support member (604) is further provided to provide radial support to the output shaft, said radial support member being adapted to rotate in said second plane.

In one embodiment of the invention several coaxial input shafts are coupled individually to several coaxial output shafts, allowing independent transmission of torque from input to output on several shafts simultaneously.

It should be appreciated that the output shafts may be coupled to a wide variety of devices, such as graspers, cutters, splicers, welders, force-feedback devices, robotic hands, and the like. In particular the use of force-feedback devices to provide a ‘return signal’ by means of one or more shafts will be found especially useful in microsurgery, robotics, and the like wherein it is desirable to have some feedback concerning the ‘feel’ of the work being done. It should be pointed out that amongst other advantages of the instant invention is the fact that the torque-providing elements that turn the input shafts may be located rather distant from the location where the torque is applied. This is especially important in such fields as arthroscopy, microsurgery, and robotics, wherein it is generally desirable that the point at which delicate operations occur are as compact as possible.

Also the presence of motors on or near joints can cause unwanted extra weight, moments of inertia, and the like. The instant invention allows many sources of torque to be transmitted in parallel in a minimum of space limited only by the shaft wall thicknesses, and at a distance from the actual operations of the output shafts that is in principle unlimited. No motors are required at the location of the joint itself, as in many current applications.

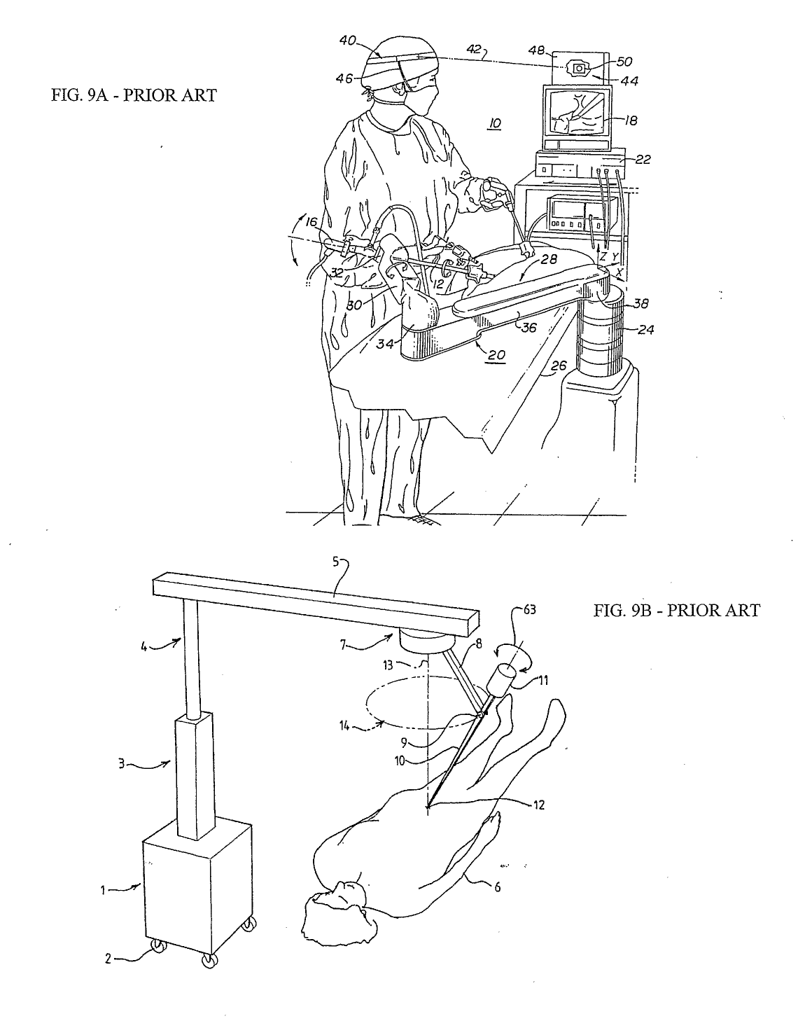

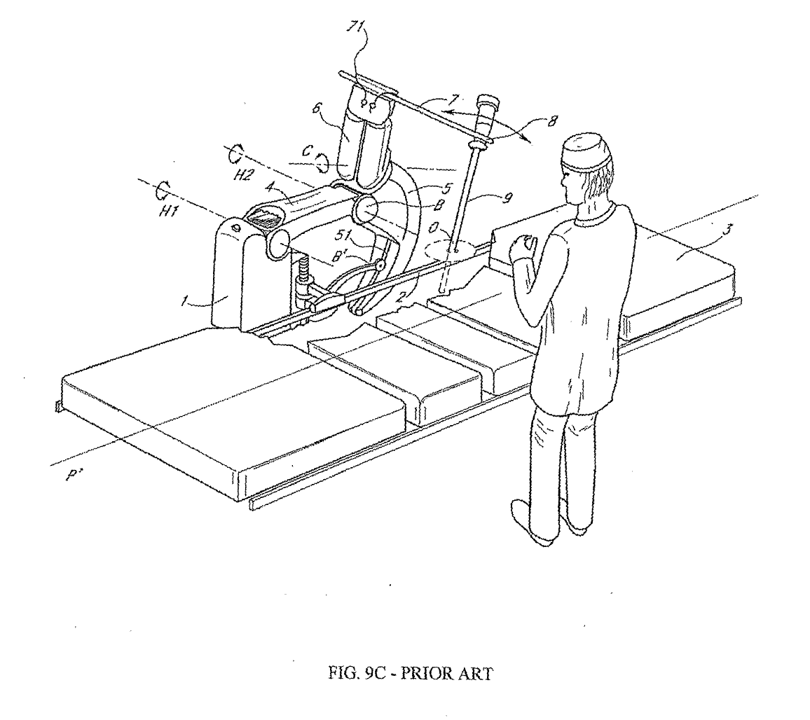

Referring to FIGS. 9a-9c it can be appreciated that the operating tool's tip could be easily replaced with a many robotic hands, splicing tool, cutting tool, welding tool, or nearly any other complex tool imaginable, requiring an arbitrary number of individual degrees of freedom.

None of which is able to provide a simple endoscope/laparoscope maneuvering system having at least 7 DOF.

Another advantage of the present invention is to further allow a single motor to activate several input shafts independently. If for example it is discovered that in a particular application certain actions requiring rotation of shaft A preclude other actions requiring rotation of shaft B, a single motor can be used to provide the torque necessary for these actions, and switched from input shaft A to input shaft B by a suitable gearbox as will be obvious to one skilled in the art.

In one embodiment of the invention access is given to the crown gears of the device, in effect changing the device into a three-terminal or ‘T’ or ‘Y’ device. In particular the central or crown gears 605, 606, 607 may be connected to input/output shafts of their own. Now more complex operations may be allowed, wherein further couplings are connected to this center shaft, or further torque sources, or further output devices such as graspers, cutters, and the like, or sensors.

We now turn to the incorporation of this coupling device into a laparoscopic instrument of improved design. In the prior art one finds a large number of laparoscopic instruments such as those shown in FIG. 9. These will in general allow a small number of degrees of freedom, the maximum found in a search of the patent literature being five degrees of freedom. In each of these cases it is apparent that ore or less complex gantries and support assemblies are required that allow freedom of motion in certain directions while restricting motion in others. The present invention does away with such requirements since each joint can be held in place or moved by means of torque from the various shafts, as described in the preceding paragraphs. Furthermore, the present invention provides, in principle, a number of degrees of freedom limited only by the number of concentric cylinders and associated gears one cares to provide in section of the device.

To improve upon this situation while keeping the simple tubular design of the laparoscope intact, we incorporate the aforementioned coupling device into an endoscope/laparoscope maneuvering system as shown in FIG. 10a,b.

The cylindrical members (consecutive arm sections) 995, 996, 997 and 998 contain a plurality of concentric cylinders, each able to rotate independently and thereby activate an independent degree of freedom. By means of these concentric cylindrical members, the couplings (i.e., the constant velocity couplers) 1002, 1003, 1004, 1005 and 1006 serve to rotate/translate the device (namely the endoscope/laparoscope 1001b or the camera 1001a) in the directions DOF1 (1007), DOF2 (1008), DOF3 (1009), DOF4 (1010), DOF5 (1011), DOF6 (1012) and DOF7 (1013).

In which DOF1 presents the ability of the system to move the endoscope or laparoscope forward and backwards in direction represented by numerical reference 1007.

DOF2 presents the ability of the system to move the endoscope or laparoscope in a zoom movement i.e. in and out of the patient body through the penetration point (represented by numerical reference 1008).

DOF3 presents the ability of the system to move the endoscope or laparoscope to the right and left in direction represented by numerical reference 1009.

DOF4 presents the ability of the system to fine tune the endoscope or laparoscope movements to the right and to the left in direction represented by numerical reference 1010.

DOF5 presents the ability of the system to fine tune the endoscope or laparoscope movements forward and backwards in direction represented by numerical reference 1011.

DOF6 presents the ability of the system to rotate the camera 1001b with respect to the endoscope's 1001a long axis. This degree of freedom is necessary to keep the horizon of the image when using endoscope with “angled edge”.

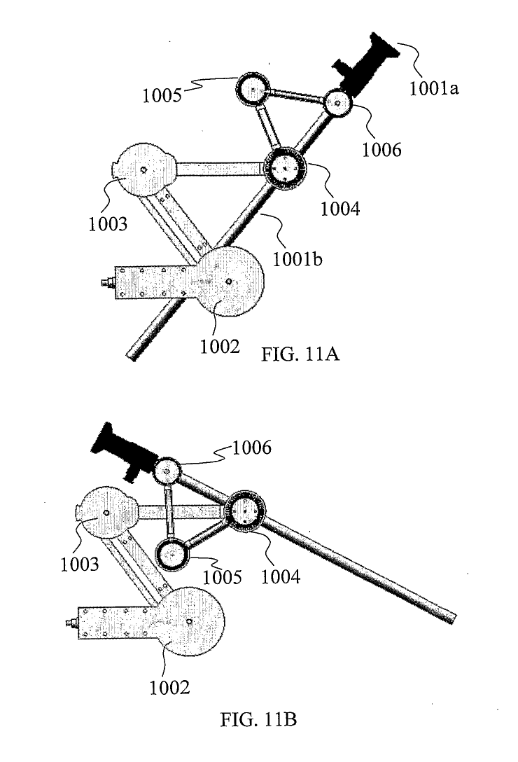

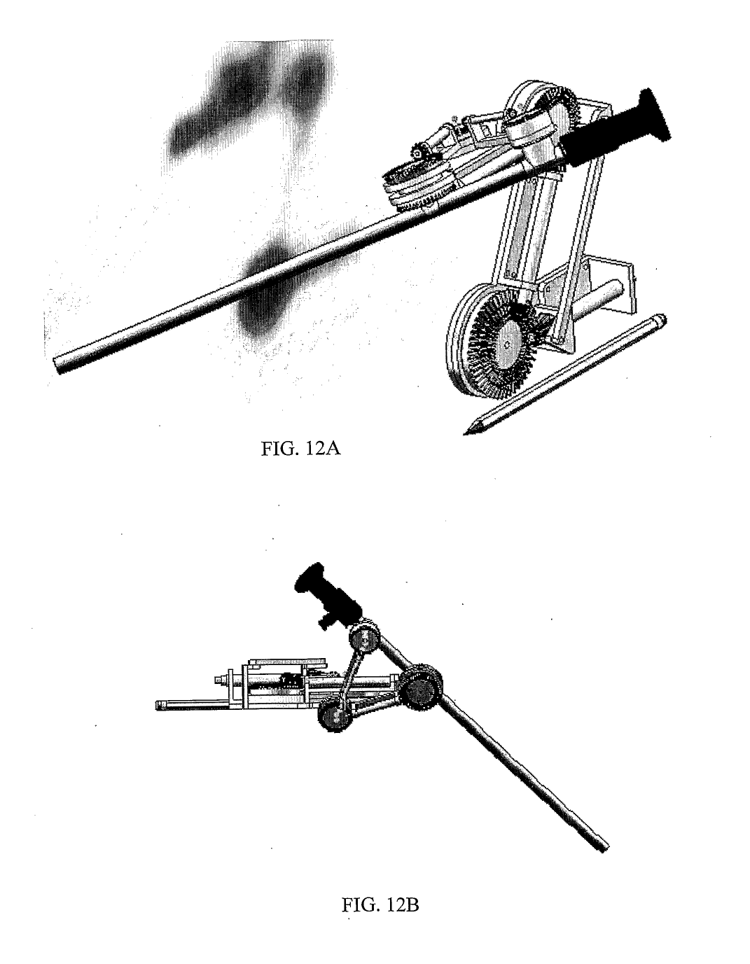

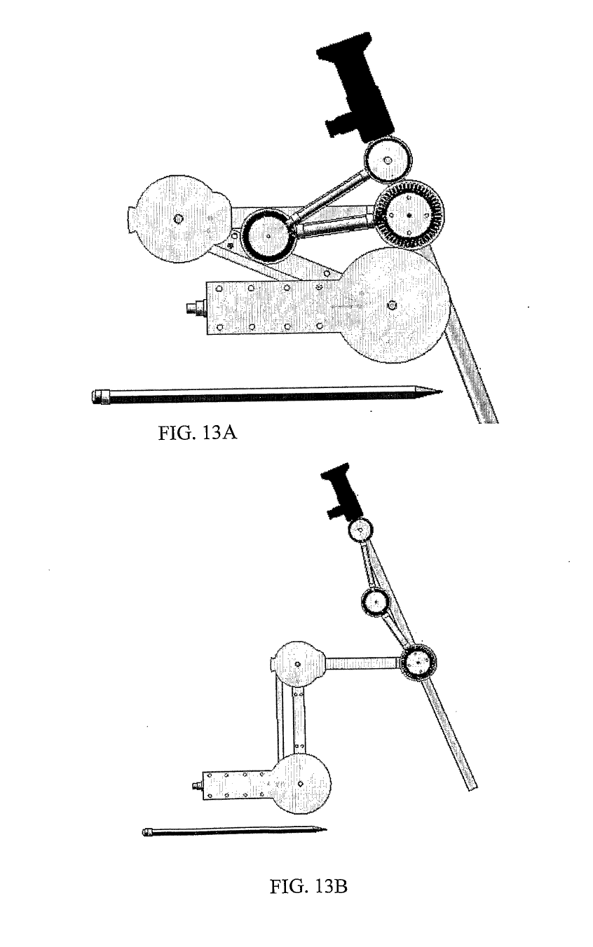

DOF7 presents the ability of the robot to rotate the endoscope 1001b about its long axis. Views of the same device from the opposite direction are shown in FIGS. 11a,b. Isometric views are shown in FIGS. 12a,b. Further side views are shown in FIG. 13a,b with a pencil shown for appreciating the small scales of the system.

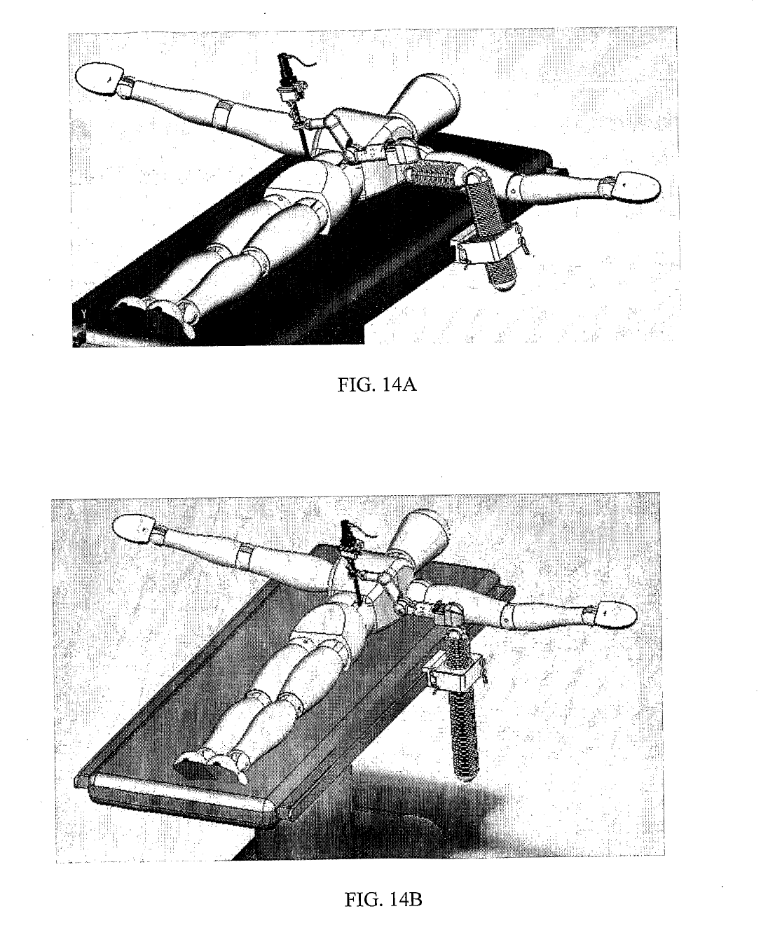

As seen in FIG. 10b, seven degrees of freedom are provided in this particular embodiment of the invention. In particular, the seven degrees of freedom are provided around (in the case of rotations) or along (in the case of translations) the following axes, which refer to a device positioned as in FIG. 14a:
a. Laparoscope/endoscope rotation (along axis 1007, or around a line perpendicular to the sagittal body plane)
b. laparoscope/endoscope translation into and out of the body—zoom in and zoom out movements (along axis 1008, or in a direction perpendicular to the axial body plane)
c. laparoscope/endoscope rotation (along axis 1009, or around a line perpendicular to the coronal body plane)
d. laparoscope/endoscope fine rotation (along axis 1010, or around a line perpendicular to the coronal body plane)
e. laparoscope/endoscope fine rotation (along axis 1011, or around a line perpendicular to the sagittal body plane)
f. camera rotation (along axis 1012, or around a line perpendicular to the axial body plane)
g. laparoscope/endoscope rotation (along axis 1013, or around a line perpendicular to the axial body plane).

As will be obvious to one skilled in the art, the camera rotation (around axis 1012) will cause a simple rotation of the view provided by the laparoscope about some point in the field of view, while the laparoscope rotation 1013 will cause a change in the area viewed by the laparoscope, as the camera or imaging apparatus viewing direction is generally not collinear with the laparoscope longitudinal direction.

In actual use such a laparoscope as described above may be operated either manually by a human being, or robotically, according to a programmed set of instructions, by a robotic mechanism obeying human commands, remotely, or the like.

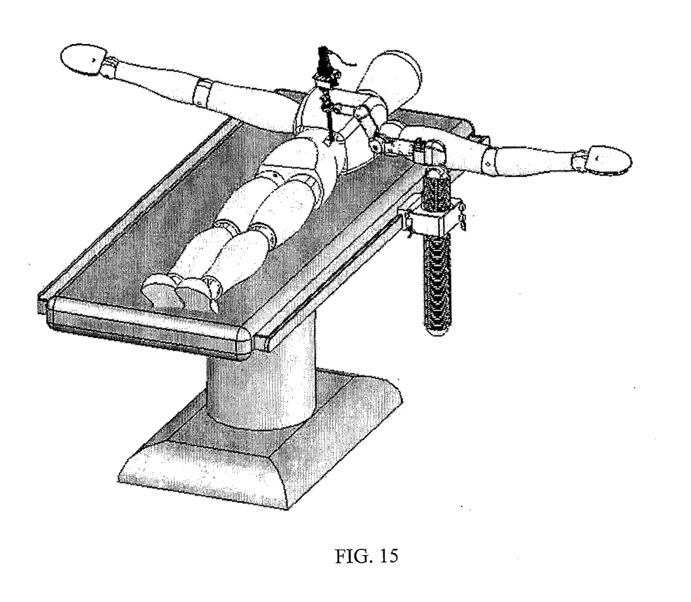

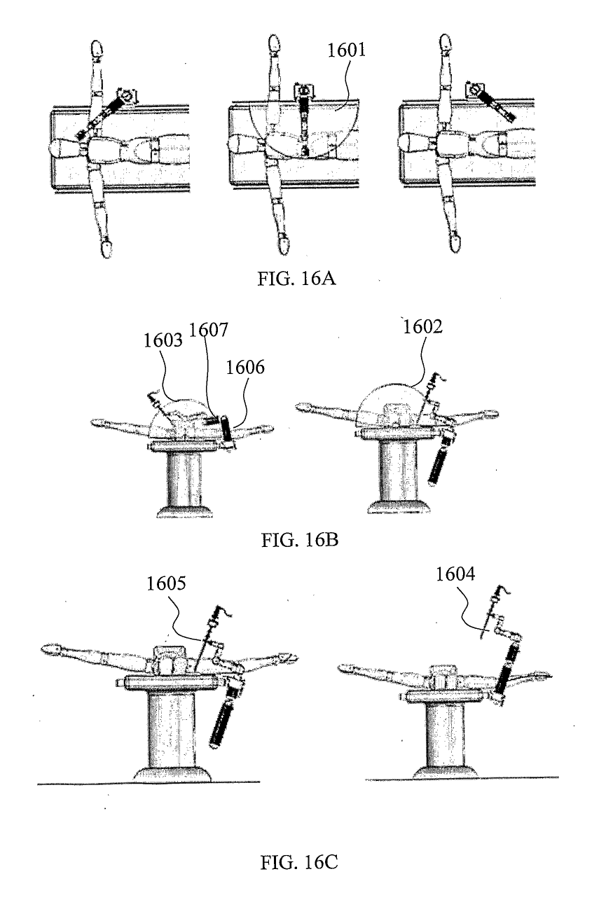

A robotic mechanism is shown in FIG. 14a,b, FIG. 15, and FIG. 16a-c. In FIG. 16a an additional degree of freedom 1601 is shown provided by the joint between arm section 1606 (FIG. 16b) and arm section 1607 that allows for rotation in the direction shown (within the coronal bodily plane). In FIG. 16b a degree of freedom is shown involving rotation within an axial bodily plane (1602, 1603), around a center of rotation centered upon the patient. In FIG. 16c the translatory degree into (1605) and out of (1604) the body is shown, equivalent to direction 1008 of FIG. 14a.

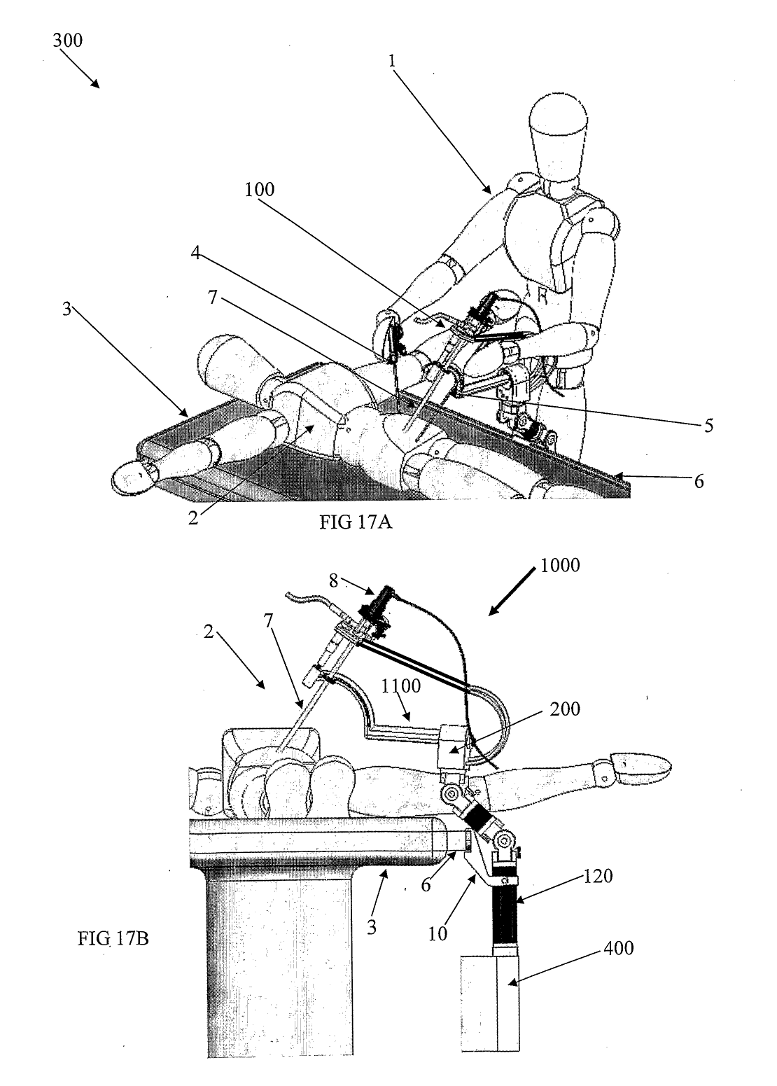

In FIG. 17a,b a human operator is shown operating a laparoscope of the current invention.

The outer arms of a laparoscope as provided in the current invention can, due to the unique angular range of the coupling employed, be folded backwards and forwards (as a parallelepiped) providing flexibility. The device can be disassembled quickly and easily.

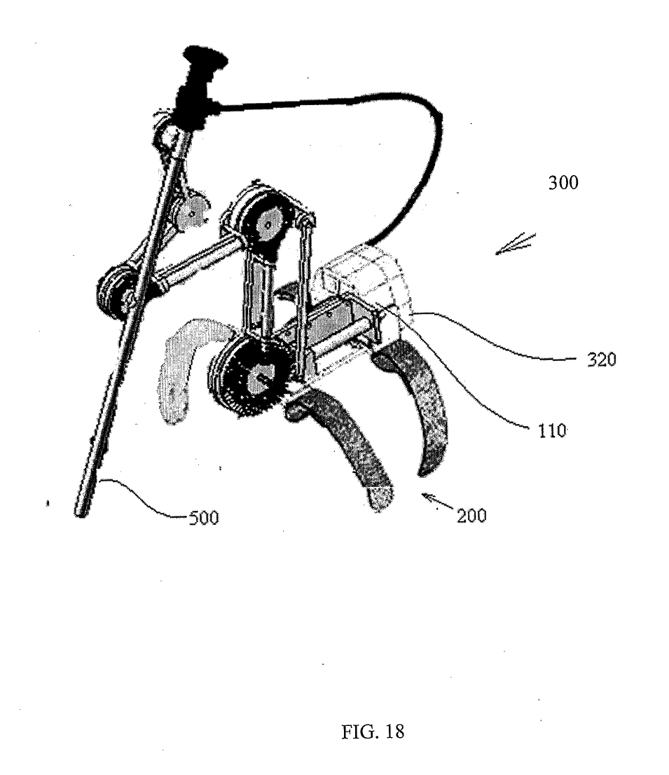

In another embodiment of the invention, the laparoscopic device of the current invention is strapped to the body of the patient by means of straps 200. Such an embodiment is shown for example in FIG. 18.

The straps 200 may be strapped e.g. to the leg or arm of a patient undergoing surgery. A positioned 110 provided with locking means 320 is used to position the straps with respect to one another.

The body gripper 200 (e.g., straps 201) basically conform the laparoscopic device to the movements of the organ (to which the straps are attached to), such that the orientation of the endoscope is adjustable accordingly to the movement of the organ.

Alternatively, the laparoscopic device is adapted to maintain a constant orientation of the laparoscope relatively to the organ (to which the straps are attached to), such that alteration in the orientation as a result of the movements of the organ is prevented.

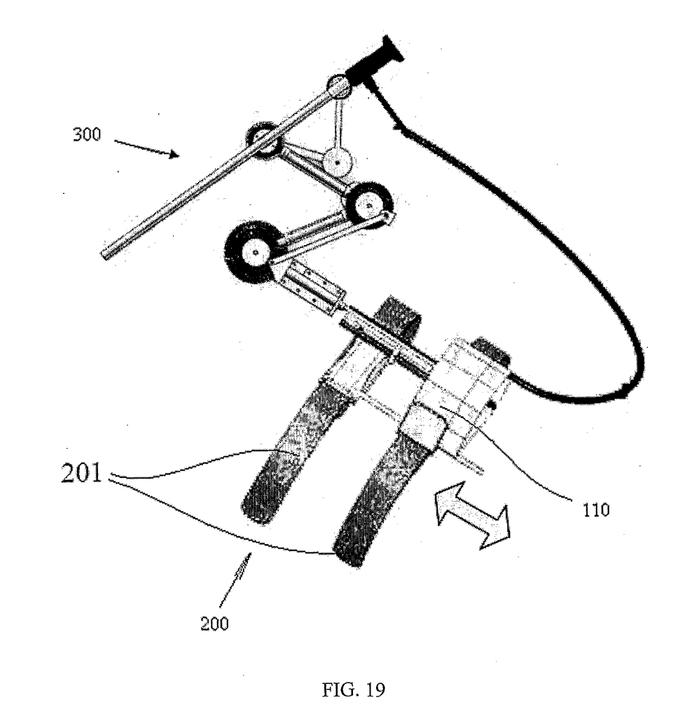

In FIG. 19 a further view of such an embodiment is shown. As described the straps 201 are used to attach the device to the body of a patient. The rest of the laparoscopic device may take any of the forms described above, taking advantage for instance of the constant-velocity coupling described. A motor box 110 is provided which is adapted to move relative to the adapter 200. This option allows the surgeon to attach the upper band of gripping bands 201 firmly to the patient limb, and then to position the mechanism 300 in the optimal arrangement relative to a joint incision (not shown) and finally fixate mechanism 300 by the lower band of gripping bands 201.

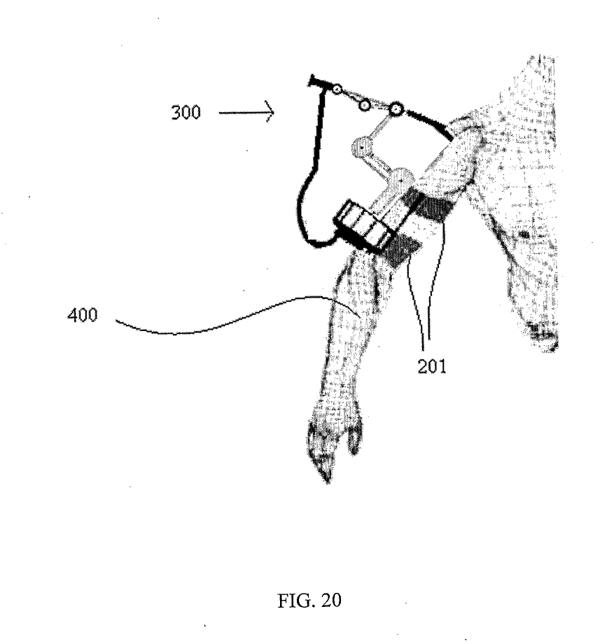

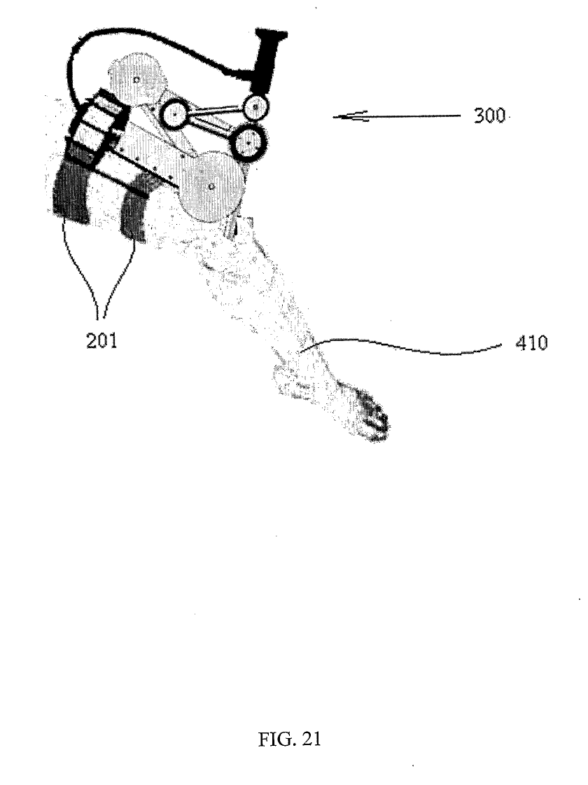

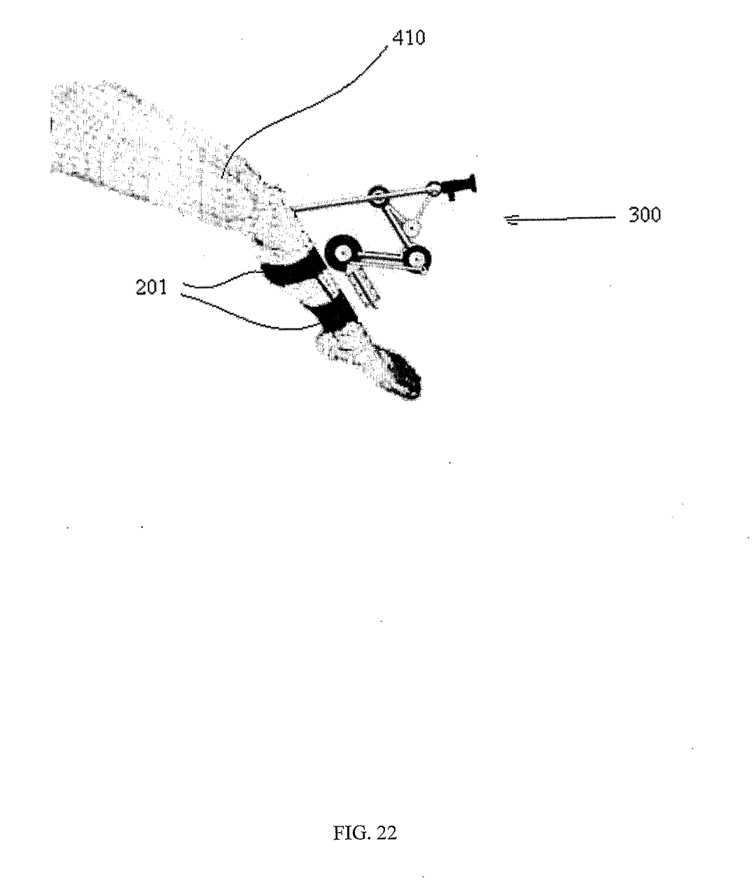

Reference is now made to FIGS. 20, 21, and 22, illustrating optional arrangements of the endoscope positioning system 300 on human limbs. The adaptor grippers 201 of body adapter 200 can embrace an arm 400 (FIG. 20), a thigh, 410 (FIG. 21) or a lower leg (FIG. 22). By using the adapter grips (201) the adapter is fixed firmly to the patient's body allowing the mechanism to move the endoscope to the desired position, even during surgeries where the body part in question must be moved. This is a common occurrence in certain surgeries, where for instance the arm must be flexed for one part of an operation and straightened for another part of the operation. Such movements are typically necessary due to the different anatomical avenues for surgery opened by the flexed arm differing from those of the straightened arm.

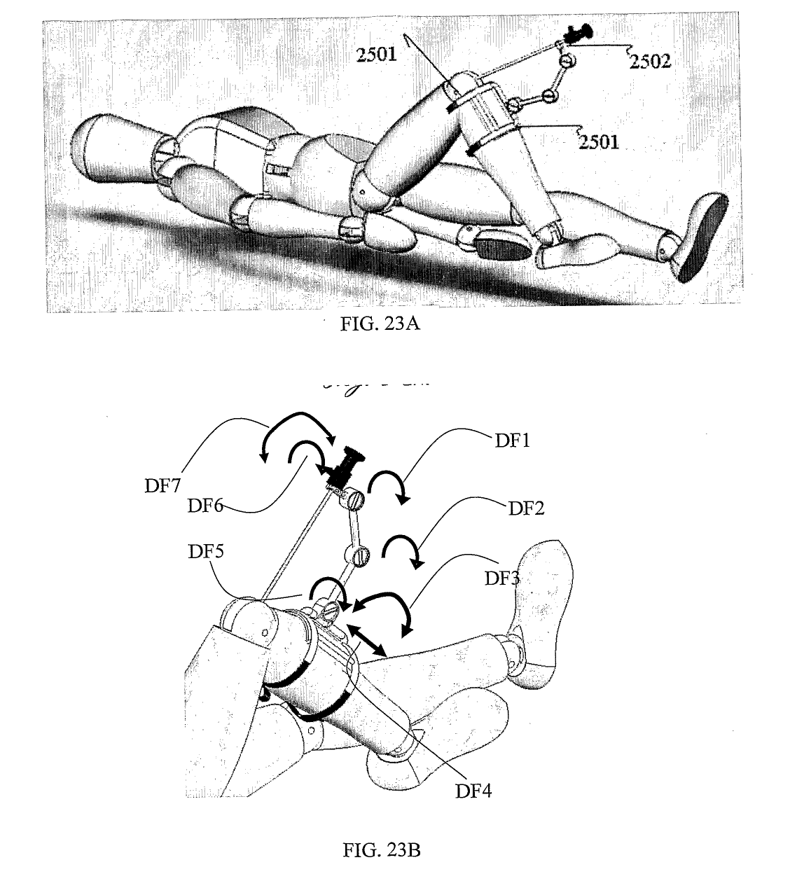

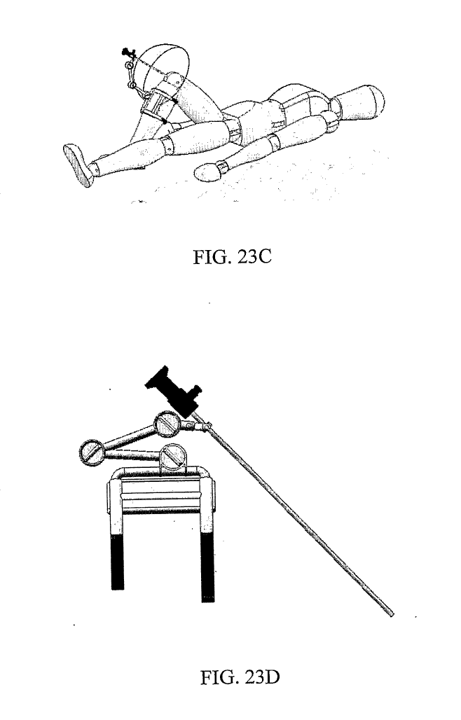

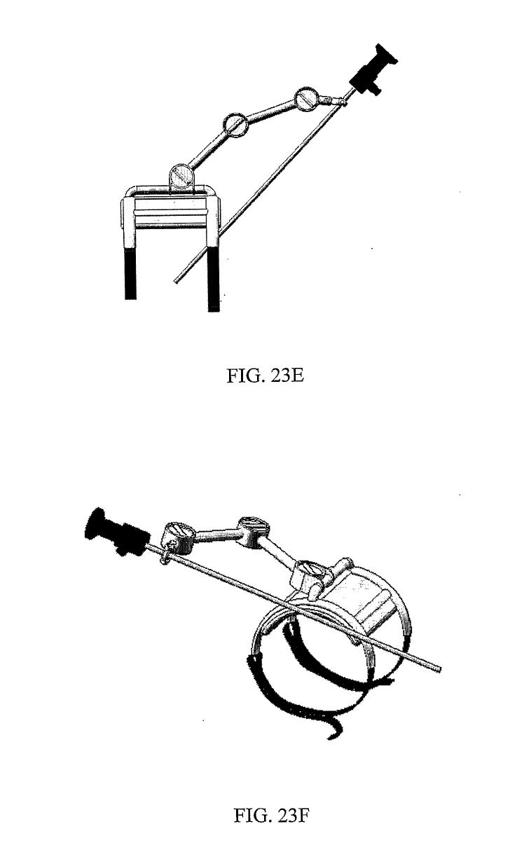

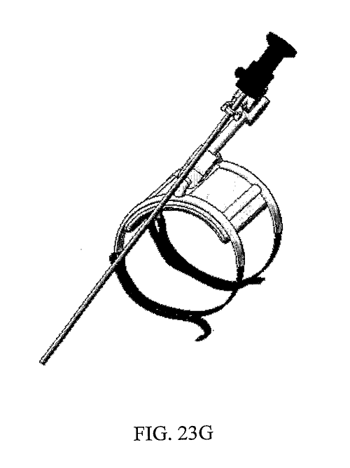

In another embodiment of the invention, the laparoscopic/endoscopic maneuvering device of the current invention is strapped to the body of the patient by means of straps. Such an embodiment is shown for example in FIGS. 23a-23g. The laparoscopic/endoscopic maneuvering device as illustrated in said figures is a non motorized device which utilizes gimbals.

The straps 2501 may be strapped e.g. to the leg or arm of a patient undergoing surgery.

A positioner 2502 is provided allowing the laparoscope/endoscope at least 8 degrees of freedom, as described above and in the figure. DF1, DF2 and DF3 represent rotation around the constant velocity couplers (i.e., the angular transmission), DF4 represents linear movement, DF5 represents rotation, DF6 represents rotational movement forwards and backwards, DF7 represents rotational movements to the left and to the right. There is another degree of freedom which is the free rotational movement of the camera around its longitudinal axis.

It is emphasized that for each device containing N constant velocity couplers (i.e., the angular transmission) there are 3+N+2 degrees of freedom.

FIG. 23b is a closer view of the laparoscopic/endoscopic maneuvering device.

FIGS. 23c-23g illustrates the wide range of motion provided by the laparoscopic/endoscopic maneuvering device.

1. A p degrees-of-freedom (DOF) laparoscope maneuverable system comprising:
a. k consecutive arm sections, each comprising n coaxial input shafts adapted to be rotated around an input axis of rotation by m sources of torque, where n and m and k are positive integers;
b. at least k−1 constant velocity couplers coupling each two of said k consecutive arm sections together, each of said constant velocity coupler comprising:
i. n coaxial input transmission means, each of which is coupled to one of said n input shafts; said input transmission means defining a first plane substantially perpendicular to said input axis of rotation;
ii. n coaxial second transmission means rotatably connected to said n input transmission means; said second transmission means rotating in a second plane, such that said second plane is substantially perpendicular to said first plane;
iii. n coaxial output transmission means rotatably connected to said n second transmission means; said output transmission means rotating in a third plane; said third plane being substantially perpendicular to said second plane;
c. n coaxial output shafts, each of which is coupled to one of said n output transmission means, said n output shafts being adapted to rotate around an output axis of rotation; such that (i) turning a given input shaft at a constant velocity will provide a constant velocity at the corresponding output shaft; and, (ii) the angle between said input axis of rotation and said output axis of rotation varies in said second plane in an angular range of about 0 to about 360 degrees;
d. at least one laparoscope coupled to at least one of said k consecutive arm sections;
wherein said p DOF are at least 7 DOF provided to said k consecutive arm sections such that said laparoscope is maneuvered.

2. The laparoscope maneuverable system according to claim 1, wherein said 7 DOF are selected from a group consisting of at least 6 rotation movements (1007, 1009, 1010, 1011, 1012, 1013, 1601, 1602), at least 1 translation movements (1008) or any combination thereof.

3. The laparoscope maneuverable system according to claim 1, wherein said input transmission means, second transmission means, and said output transmission means are selected from a group consisting of gearwheels, wheels, crown gears, bevel gears, spur gears, belts, and combinations thereof.

4. The laparoscope maneuverable system according to claim 1, additionally comprising:
a. an axial support member (601) adapted to provide axial support to said n output shafts in said third plane; and,
b. a circular track (618) centered on the axis of rotation of said second transmission means, said axial support member being adapted to fit into said track and slide within it.

5. The laparoscope maneuverable system according to claim 1, additionally comprising a radial support member (604) adapted to provide radial support to said n output shafts, said radial support member being adapted to rotate in said second plane.

6. The laparoscope maneuverable system according to claim 1, wherein the gear ratio between said input and output shafts is between about 10 and about 0.1.

7. The laparoscope maneuverable system according to claim 1, additionally comprising n coaxial auxiliary shafts in rotating communication with said n second transmission means, said n coaxial auxiliary shafts rotating in said second plane, and said n coaxial auxiliary shafts capable of either being driven by said input shafts or driving said input shafts.

8. The laparoscope maneuverable system according to claim 1, additionally comprising locking means adapted for preventing relative movement between one or more of said input axis shafts and said constant velocity joint, wherein said constant velocity joint is caused to rotate as a body with said locked input axis shafts.

9. The laparoscope maneuverable system according to claim 1, additionally comprising locking means for preventing relative movement between one or more of said output axis shafts and said constant velocity joint, wherein said constant velocity joint is caused to rotate as a body with said locked output axis shafts.

10. A method of maneuvering a surgical instruments during laparoscopic surgery whilst providing p Degrees of Freedom (DOF), comprising steps of:
a. providing k consecutive arm sections, each comprising n coaxial input shafts adapted to be rotated around an input axis of rotation by m sources of torque, where n and m are positive integers;
b. providing at least k−1 constant velocity couplers coupling said consecutive arm sections, each said constant velocity coupler adapted to be rotated around an input axis of rotation by m sources of torque, where n and m are positive integers and each said constant velocity coupler comprising:
i. n coaxial input transmission means, said input transmission means defining a first plane substantially perpendicular to said coaxial axis;
ii. n coaxial second transmission means rotatably connected to said n input transmission means; said second transmission rotating in a second plane, such that said second plane is substantially perpendicular to said first plane;
iii. n coaxial output transmission means rotatably connected to said n second transmission means; said output transmission means rotating in a third plane; said third plane being substantially perpendicular to said second plane;
c. providing n coaxial output shafts, said n output shafts being adapted to rotate around an output axis of rotation, and said n output shafts being coupled to said n coaxial output transmission means, whereby turning a given input shaft at a constant velocity will provide a constant velocity at the corresponding output shaft, and furthermore wherein the angle between said input axis of rotation and said output axis of rotation varies in said second plane in an angular range of about 0 to about 360 degrees;
d. coupling said k consecutive arm sections together with said k−1 couplers;
e. providing at least one surgical instrument;
f. coupling said surgical instrument to said output shafts; and,
g. maneuvering said surgical instrument by means of rotating one or more of said coaxial input shafts thereby providing said p DOF to said k consecutive arms and said surgical instrument,
wherein said step of proving said p DOF comprises at least 7 DOF provided to said k consecutive arm sections.

11. A method for transmitting torque to surgical instruments during laparoscopic surgery comprising steps of:
a. providing k consecutive arm sections, each comprising n coaxial input shafts adapted to be rotated around an input axis of rotation by m sources of torque, where n and m are positive integers;
b. providing at least k−1 constant velocity couplers coupling said consecutive arm sections together, said constant velocity couplers comprising:
i. n coaxial input transmission means, each of which is coupled to one of said n input shafts; said input transmission means rotating in a first plane substantially perpendicular to said coaxial axis;
ii. n coaxial second transmission means rotatably connected to said n input transmission means; said second transmission means rotating in a second plane, such that said second plane is substantially perpendicular to said first plane;
iii. n coaxial output transmission means rotatably connected to said n second transmission means; said output transmission means rotating in a third plane; said third plane being substantially perpendicular to said second plane;
c. n coaxial output shafts, each of which is coupled to one of said n output transmission means, said n output shafts being adapted to rotate around an output axis of rotation, whereby turning a given input shaft at a constant velocity will provide a constant velocity at the corresponding output shaft, and furthermore wherein the angle between said input axis of rotation and said output axis of rotation varies in said second plane in an angular range of about 0 to about 360 degrees;
d. providing at least one surgical instrument;
e. coupling said surgical instrument to said output shafts; and,
f. performing surgical functions by means of rotating one or more of said coaxial input shafts thereby providing said p DOF to said k consecutive arms and said surgical instrument;
wherein said step of proving said p DOF comprises at least 7 DOF provided to said k consecutive arm sections.

12. The method according to claim 11, additionally comprising step of selecting said 7 DOF from a group consisting of at least 6 rotation movements (1007, 1009, 1010, 1011, 1012, 1013, 1601, 1602), at least 1 translation movements (1008) or any combination thereof.

13. The method according to claim 11, wherein said input transmission means, second transmission means, said output transmission means are selected from a group consisting of gearwheels, wheels, crown gears, bevel gears, spur gears, belts, or any combination thereof.

14. The method according to claim 11, additionally comprising
a. an axial support member (601) adapted to provide axial support to said n output shafts in said third plane; and,
b. a circular track (618) centered on the axis of rotation of said second transmission means, said axial support member being adapted to fit into said track and slide within it.

15. The method according to claim 11, additionally comprising a radial support member (604) adapted to provide radial support to said n output shafts, said radial support member being adapted to rotate in said second plane.

16. The method according to claim 11, providing a gear ratio between said input and output shafts is between about 10 and about 0.1.

17. The method according to claim 11, additionally comprising the step of providing n coaxial auxiliary shafts in rotating communication with said n second transmission means, said n coaxial auxiliary shafts rotating in said second plane, and said n coaxial auxiliary shafts either being driven by said input shafts or driving said input shafts.

18. The method to claim 11, additionally comprising locking means adapted for preventing relative movement between one or more of said input axis shafts and said constant velocity joint, wherein said constant velocity joint is caused to rotate as a body with said locked input axis shafts.

19. The method according to claim 11, additionally comprising locking means adapted for preventing relative movement between one or more of said output axis shafts and said constant velocity joint, wherein said constant velocity joint is caused to rotate as a body with said locked output axis shafts.

20. A laparoscopic instrument characterized by p degree-of-freedom (DOF) comprising:
a. k consecutive arm sections, each comprising n coaxial input shafts adapted to be rotated around an input axis of rotation by m sources of torque, where n and m are positive integers;
b. at least k−1 constant velocity couplers coupling each two of said consecutive arm sections together, each of said constant velocity couplers comprising:
i. n coaxial input transmission means, each of which is coupled to one of said n input shafts; said input transmission means defining a first plane substantially perpendicular to said input axis of rotation;
ii. n coaxial second transmission means rotatably connected to said n input transmission means; said second transmission means rotating in a second plane, such that said second plane is substantially perpendicular to said first plane;
iii. n coaxial output transmission means rotatably connected to said n second transmission means; said output transmission means rotating in a third plane; said third plane being substantially perpendicular to said second plane;
c. n coaxial output shafts, each of which is coupled to one of said n output transmission means, said n output shafts being adapted to rotate around an output axis of rotation, whereby turning a given input shaft at a constant velocity will provide a constant velocity at the corresponding output shaft, and furthermore wherein the angle between said input axis of rotation and said output axis of rotation varies in said second plane in an angular range of about 0 to about 360 degrees;
d. at least one laparoscopic instrument coupled to at least one of said k consecutive arm sections, adapted for performing surgical functions;
wherein said p DOF comprise at least 7 DOF provided to said surgical tools

21. A non motorized laparoscopic/endoscopic maneuvering device comprising:
a. at least two consecutive arm section;
b. at least one gimbal at least coupling each two of said consecutive arm sections together; and,
c. at least one laparoscope coupled to said consecutive arm;
wherein said laparoscopic/endoscopic maneuvering device maneuvers said laparoscopic/endoscopic in a non motorized manner.
